(19) United States
(12) Patent Application Publication
(10) Pub. No.: US 2025/0049008 A1
(43) Pub. Date: Feb. 13, 2025

(54) INSTALLATION FOR TREATING ARTHROPOD LARVAE, IN PARTICULAR INSECTS AND MORE SPECIFICALLY DIPTERA LARVAE

(71) Applicant: Innovafeed, Nesle (FR)

(72) Inventors: Elise Gosset, Paris (FR); Bastien Oggeri, Paris (FR); Audrey Schuller, Paris (FR); Martin Pascal, Paris (FR)

(21) Appl. No.: 18/723,673

(22) PCT Filed: Dec. 23, 2022
(86) PCT No.: PCT/EP2022/087816

§ 371 Date: Jun. 24, 2024

(30) Foreign Application Priority Data
Dec. 24, 2021 (FR) 2114497

(51) Int. Cl. A01K 67/033; A23K 10/20

(52) U.S. Cl. CPC A01K 67/033; A23K 10/20; A01K 2227/706

(57) ABSTRACT

An installation for treating arthropod larvae, in particular, insects, and more specifically, ground Diptera larvae, comprises at least one piece of equipment producing glue water, which is extracted from the mixture of ground, larvae. The installation comprises a glue water circuit supplying a ground larvae treatment equipment comprising a circuit for diluting the ground larvae and/or a glue water evaporator and/or a meal dryer.

### CROSS REFERENCE TO RELATED APPLICATIONS

This application is a national phase entry under 35 U.S.C. § 371 of International Patent Application PCT/EP2022/087816, filed Dec. 23, 2022, designating the United States of America and published as International Patent Publication WO 2023/118604 Al on Jun. 29, 2023, which claims the benefit under Article 8 of the Patent Cooperation Treaty to French Patent Application Serial No. FR2114497, filed Dec. 24, 2021.

### TECHNICAL FIELD

The present disclosure relates to the field of industrial farming of arthropods, in particular, of insects, and more specifically, Diptera for the purposes of food production.

The present disclosure relates more particularly to the field of arthropod farming, specifically insects, and more specifically, Diptera, in particular, the black soldier fly.

### BACKGROUND

Arthropods have a certain number of characteristics that make them well suited to use in animal feed. Arthropods contribute a high protein content, while being rich in other beneficial nutrients such as fats, minerals and vitamins. The levels of protein concentration in insect meals intended for animal feed vary between 55% and 75%. The insects are characterized by a higher food conversion rate and can therefore become a very valuable feed source for farm animals. Insects are a natural component of the feed for animals such as carnivorous fish and poultry (for example, insects can provide up to 70% of the food needs of trout).

Moreover, these products also have a well-balanced nutritional profile to meet human food needs.

These considerations have led to the development of the large-series automated production of food from arthropod farming, and more particularly insects, in industrial sites organized into complementary spaces specialized in the egg-laying, hatching, rearing and collection of mature animals and the treating thereof to extract the compounds of interest.

These industrial sites must be optimized to allow the industrialization of large volumes of larvae. One of the most critical stages is the treating of live larvae, in particular, those of the black soldier fly, Hermetia illucens. The larvae are processed into protein meal and quality oil. The rearing medium wherein the larvae evolve at the end of their growth cycle, called frass, is also recycled. Frass consists of a mixture of larva droppings and residue from the uneaten substrate, dried and fermented.

This treating stage comes after the stages of egg production under industrial conditions, collection of eggs laid by female insects, and concentration of larvae for rearing, as they need to be grouped together as homogeneously as possible, in batches of neonate larvae all at the same stage of maturity in a given batch. Generally, egg-laying is carried out in a cage confining the flies in an enclosed space wherein collectors having laying surfaces, for example, grooved plates, are arranged, on which the females deposit the eggs. These collectors are recovered to allow the eggs to hatch into neonate larvae, which are then injected onto a nutrient medium in insect-raising modules. These neonate larvae are then raised in multi-stage growth modules to reach maturity.

These larvae are finally devitalized and transformed into a nutrient-rich pulp by grinding and heat treatment. This pulp can then be subjected to additional treatments, and finally undergoes an extraction stage for the compounds of interest, in particular, proteins and oily substances, using an installation that is the subject of the present disclosure.

Various solutions for separating compounds of interest from pastes obtained from larvae are known in the prior art.

The patent application WO2020011903 describes a method for obtaining food and/or feed industry products from insects, and describes an installation comprising a three-way decanting system. The insect pulp is divided into a fatty aqueous phase on a first outlet and a solid phase on a second outlet. The fat-containing aqueous phase can then be divided into an aqueous phase and a fat phase in a separator. This document specifies that the aqueous phase can be subjected to drying, particularly in an evaporator.

Patent application WO2014123420 describes a method to convert insects or worms into nutrient streams, such as a fat-containing fraction, an aqueous protein-containing fraction and a solid-containing fraction. The method comprises the steps of: (a) squashing insects or worms to obtain a pulp, wherein the insects or worms are reduced in size, (b) heating the pulp to a temperature of 70-100° C. and (c) subjecting the heated pulp to a physical separation step, preferably decanting and/or centrifuging.

Patent CN208843920 describes a method for collecting greasy filth and treating it with a three-phase separating device, a greasy filth agitator tank, and a two-phase laminated flow device. It is equipped with a blender and heater in the greasy filth collecting pit, and adds a cleaning agent to the greasy waste, sludge and water, and the sludge mouth is connected to the greasy sludge agitator tank.

Patent KR20200040330 relates to a method for producing insect larva oil with improved palatability, which comprises the steps of:
(a) extraction of oil from insect larvae;
(b) acid treatment;
(c) urea treatment; and
(d) addition and reaction of an adsorbent.

Patent application WO2013191548 relates to a method to convert insects or worms into nutrient streams, such as a fat-containing fraction, an aqueous protein-containing fraction and a solid-containing fraction. The method comprises the steps of: (a) squashing insects or worms to obtain a pulp, (b) subjecting the pulp to enzymatic hydrolysis to obtain a hydrolyzed mixture, (c) heating the hydrolyzed mixture to a temperature of 70-100° C., and (d) subjecting the mixture to a physical separation step, preferably by decanting and/or centrifuging.

Prior art solutions often require complex installations that are prone to breakdowns or fouling, due to the multiple steps involved in transferring intermediate compounds from one treating station to the next via transfer mechanisms that have to operate in unfavorable environments (humidity, sticky materials, etc.).

Many of these solutions also require substantial water and energy consumption.

### BRIEF SUMMARY

The present disclosure relates to an installation for treating arthropod larvae, in particular, insect larvae, and more specifically, ground Diptera larvae.

The present disclosure also concerns an installation for treating arthropod larvae, in particular, insect larvae, and more specifically, ground Diptera larvae, comprising a decanting system for separating the solid and liquid phases of the ground larvae, having three outlets:
the solid phase outlet is connected to a meal dryer,
a first liquid outlet feeds a glue water circuit, and
a second liquid outlet feeds the oil collection circuit,

wherein the glue water circuit supplies a ground larvae treatment equipment comprising a circuit for diluting the ground larvae and/or a glue water evaporator and/or a meal dryer.

Advantageously, the system also has the following features:
the solid phase outlet is directly connected to a meal dryer via a chute located below the outlet of the decanting system, allowing three phases to be separated for gravity transfer;
the meal dryer operates under vacuum;
the meal dryer comprises upstream a sealed transfer lock formed by two valves that open and close alternately to maintain the vacuum in the meal dryer;
the vacuum meal dryer is isolated from the parts of the method downstream by a sluice forming a seal;
the meal dryer comprises, at the outlet, disks equipped with extraction scoops to direct the meal toward the outlet; and
the meal dryer is also supplied with concentrate from a glue water evaporator and/or glue water from a three-phase separation decanting system, as well as an antioxidant injection line.

Advantageously, the installation includes a condenser comprising a plate heat exchanger for condensing the vapors evaporated from the meal during drying, and a means for discharging the condensates.

Advantageously, the system also has the following features:
it includes equipment for concentrating glue water to 30-50% dry matter to form a concentrate that is reinjected upstream of a meal dryer to be mixed with the cake from the three-phase separation decanting system and dried in the meal dryer;
it comprises a three-way valve controlling the flow of oil from the second liquid outlet to a recycle tank upstream of the three-phase separation decanting system or to a buffer tank, the valve being controlled by means for measuring the quality of the oil from the three-phase separation decanting system;
the means for measuring the quality of the oil coming from the three-phase separation decanting system is a turbidimeter; and
it comprises thermal regulation means to ensure that the temperature of the ground larvae pulp at the inlet to the three-phase separation decanting system is between 80° C. and 95° C.

### BRIEF DESCRIPTION OF THE DRAWINGS

Other features and advantages will become apparent from the following description of the present disclosure, given only by way of example, referring to the accompanying drawings wherein:

FIG. 1 shows the diagram of treatments to prepare the pulp subjected to separation treatments according to the present disclosure;

FIG. 2 shows a diagram of separation treatments according to the present disclosure;

FIG. 3 shows a schematic view of a three-phase decanter used for separation according to the present disclosure;

FIG. 4 shows a schematic view of a hammer mill according to the present disclosure.

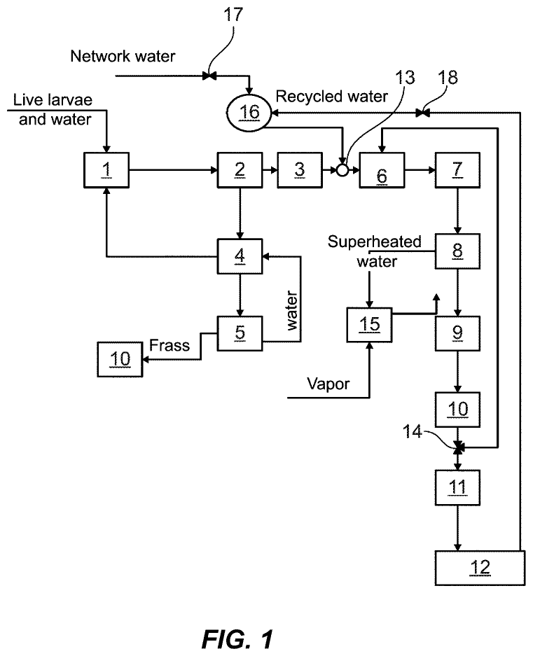

### DETAILED DESCRIPTION

The present disclosure concerns the recovery of compounds of interest from a pulp of ground larvae obtained after the application to arthropod larvae, preferably insects and more particularly Diptera, in particular, black soldier flies, of devitalization treatments for live larvae, and hygienization of this pulp when the method chosen for devitalization is carried out at temperatures too low to ensure the destruction of all bacteria. Hygienization of this pulp can be carried out in a hygienization heat exchanger whose function is to raise the product to the target temperature for heat treatment, then in a chamber that keeps the product at the right temperature for the desired time.

The treatment of this pulp, which is the subject of the present disclosure, consists, in particular, in separating it into three phases: a solid phase, a heavy liquid phase and a light liquid phase.

### Pretreatment of Mature Live Larvae

Prior to this separation covered by the present disclosure, the mature larvae are subjected to one or more pretreatments consisting in devitalizing them by heat treatment in an aqueous medium, grinding the devitalized larvae and mixing them with water to prepare a nutrient pulp, then sanitizing this pulp.

Prior to the devitalization stage, the larvae are usually separated from the frass (a mixture of larval droppings and uneaten, dried and fermented substrate residues) via two particle size separation stages. The first step is to use a trampoline strainer to separate the live larvae from the powdery frass. The second stage uses a circular screen to separate live larvae from any agglomerated frass pellets.

The larvae fall by gravity into a parallelepiped tank, avoiding the need for complex installations requiring pumps or motorized movement systems. The larvae are mixed with water in a proportion controlled according to the larvae flow rate measured on the weighing belt. Water is used for many purposes.
Firstly, water is used as a transport medium, making it easier to handle and transfer larvae by pumping.
The water also serves as a thermal vector to thermally devitalize the larvae.
The water is still used to finish cleaning the larvae and constitutes a third purification stage. Larvae are relatively moist on the surface, and “grains” of frass can stick to them. In this case, sieving with the trampoline strainer is ineffective, as the stuck frass remains attached to the larvae. In water, frass can detach and pass into the aqueous phase.

Live larvae and residual frass are immersed in a tank containing hot water. The contents of this tank, the mixture of larvae, water and residual frass, are transferred to an exchanger (1) by way of a membrane pump to bring the mixture to a temperature of between 55° C. and 95°° C. (preferably between 75° C. and 80° C.) in order to devitalize the larvae. The mixture of devitalized larvae, water and residual frass is then poured onto a strainer (2), separating the devitalized larvae from the loaded effluent (water and residual frass). The larvae are transferred to a mill (3). The loaded effluent is discharged into the dirty compartment of a decanting tank (4), separating the frass that is transferred to a frass press (5) and then to a frass recovery unit (10). The purified water obtained at the outlet of the frass press can then be reinjected into the clean compartment of the decanting tank to concentrate the residual frass in the dirty compartment. Clean water is also drawn from the clean compartment of the decanting tank and re-injected into the mixing tank.

The devitalized and cleaned larvae from the strainer (2) are transferred to two mills in series (3) using an eccentric rotor pump.

### Transfer of Devitalized Larvae Pulp

An eccentric rotor pump (7) transfers the ground larvae from the 4 m³ launch tank (6), fed by the mills (3), through a heat exchanger (8) and then a chamber (9) and cooler (10) to a buffer tank (11) with a capacity of 15 m³, for example.

### Larvae Dilution

Just upstream of the second of the two mills in series (3), water is introduced to dilute the larvae. The larvae flow rate is measured by an electromagnetic flowmeter (13) at the entrance to the treating zone, upstream of the water addition. Water flow is also measured by an electromagnetic flowmeter (16). A dilution ratio is maintained.

Depending on the actual flow rate of ground larvae, the water flow rate is adjusted to maintain the target dilution ratio (between 20% and 50%). The dilution ratio is chosen in relation to the quality of phase separation in the three-phase separator (12) and to pressure drops in the pipes and equipment. The ratio can be set by the operator.

The water added to the larvae comes from two different sources.
On the one hand, the water used is glue water recovered at the outlet of the three-phase decanter (12). The temperature of the glue water is over 70° C. The added glue water comes directly from the three-phase decanter and is therefore part of the same production batch.
On the other hand, the water used comes from a PHW network (Process Hot Water, a network distributing hot water throughout the industrial site from the heating of softened water). The temperature of the PHW is 55° C.

Two pilot valves (17, 18) control the respective flow rates of these two sources to optimize energy consumption. When the line is started, water from the PHW network is added to the larvae upstream of the second mill (3). The changeover from the PHW network to the glue water produced by the three-phase decanter (12) takes place at the end of the line start process.

The use of the glue water produced by the three-phase decanter (12) makes it possible to:
optimize energy by mixing hot water at around 70° C. with ground larvae; and
optimize water consumption.

When using PHW, water is added to the system, whereas when using glue water, no additional water is introduced into the system, thus reducing the amount of glue water produced. This also represents significant energy optimization since the glue water is evaporated and then dried.

### Separation of Ground Larvae Pulp

After the various treatments required to transform live larvae into a hygienized larvae pulp (cleaned of frass residues), the substances of interest present in this pulp are then separated.

Referring to FIG. 3, the main separation stage is carried out via a centrifugal three-phase decanter (12) comprising a rotating bowl (30) at the center of which a screw (31) is also rotating. It mechanically separates the pulp formed by ground larvae and water introduced via a feed duct (35) from the bowl (30), into three phases.
Phase 1: the defatted cake, which is still about 50% moist. It is sent to a vacuum disk dryer and discharged by gravity via a first outlet (32).
Phase 2: glue water, a liquid loaded with soluble proteins, which is discharged via a pipe (33) into a recovery tank connected to a lifting pump.
Phase 3: the oil, very pure (<1% impurities), flows from the bowl (30) and is sent to the conditioner via a pipe (34).

The temperature at the inlet to the three-phase decanter (12) is at least 80° C. for effective separation of the three phases, and preferably less than 95° C.

The bowl speed and the differential speed, i.e., the difference between the speed of the bowl (30) and that of the screw (31), are adjusted to target a yield/purity compromise and:
maximize protein recovery from the cake and glue water;
maximize protein purity to obtain the best possible protein content for defatting the cake and glue water; and
secondarily, to limit moisture in the cake to reduce the energy costs of drying the cake.

One solution is to start with maximum bowl (30) speed and adjust the differential speed to find the best purity/yield compromise.

Another way of optimizing the separation of oil and glue water, and thus minimizing the amount of water in the oil, is to manually adjust a turbine on the three-phase decanter to position the oil/water interface on the machine's internal separation disk.

The parameters are preset at start-up and can then be controlled according to the quality analysis of the products separated by the three outlets (32 to 34) of the three-phase decanter, in particular, according to moisture and fat content.

### Treating the Meal

The term “meal” refers to the cake from the three-phase decanter to which glue water or the concentrate from the evaporation of glue water may be added, and which is then dried and ground.

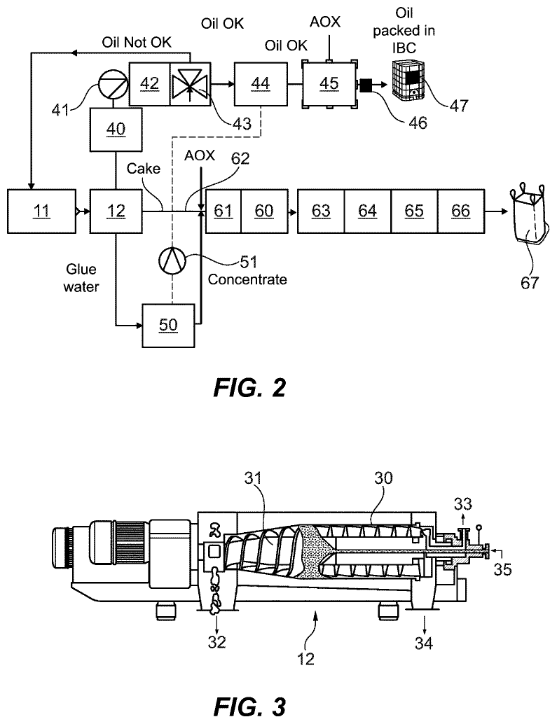

Referring to FIG. 2, the cake leaving the three-phase decanter (12) falls by gravity into a chute (62) located between the solids outlet of the three-phase decanter and the dryer feed (60). As the cake is a difficult product to handle, and handling equipment is difficult to clean, the gravity method ensures efficient and economical transfer between the decanter (12).

The meal dryer (60) operates under vacuum. Between the three-phase decanter (12) and the dryer (60), two valves (61) open and close alternately to maintain the vacuum in the dryer (60). These two valves (61) are never open at the same time, forming a sealed transfer lock. This chute (62) also carries two other products:
the concentrate leaving the evaporator (50) or glue water leaving the three-phase decanter (12); and
an antioxidant injection.

The addition of glue water or glue water concentrate is not essential for meal production, but it does improve meal quality.

### Adding Antioxidant to the Meal

The antioxidant is added before the meal is dried, to ensure that the meal keeps well. Passage through the drier (60) ensures that the antioxidant is thoroughly mixed with the meal.

The antioxidant concentration in meal after drying is set at 800 ppm. An antioxidant feed pump pumps antioxidant from an antioxidant canister to the chute (62) between the three-phase decanter (12) and the dryer (60). The flow rate of the antioxidant pump is set by a programmable logic controller based on the defined concentration and the dryer (60) inlet flow rate. The glue water flow rate and concentrate flow rate are measured by flowmeters. The cake flow rate is estimated by the programmable logic controller based on the ground larvae pulp flow rate at the inlet to the three-phase decanter (12).

### Drying the Meal

The cake leaving the three-phase decanter (12) still contains 50% moisture. Similarly, the glue water concentrate is also between 50% and 70% moisture. A drying step performed by a vacuum disk dryer (60), comprising a rotor on which disks rotate in a closed enclosure. These disks feature the scoops described below.

Steam from the network circulates through the discs to heat them. The steam condenses and the steam condensate leaving the dryer is sent to the boiler via the condensate network. There is no contact between the steam and the meal.

The meal circulates between the discs. Scoops located on the disks allow the meal to advance at higher or lower speeds, depending on the shape of the scoops.

The moisture content of the meal decreases as the product moves through the dryer. The input moisture content is around 50-60%, while the output meal moisture content is between 5% and 7%. Disk speed is continuously monitored.

A sampling point is located at the dryer outlet (60). Meal samples are taken by the operator every 10 or 20 minutes for off-line moisture analysis on a moisture analyzer. When humidity drops below 10%, samples are taken every 5 minutes. Alternatively, a humidity measurement sensor located in the dryer at the outlet enables continuous humidity monitoring. Meal extraction can then take place automatically according to humidity.

Once the target humidity of between 5% and 7% has been reached, the operator opens the valve located on the meal outlet of the dryer (60). The shape of the scoops on the disks at the outlet directs the meal toward the outlet on the side of the dryer. The scoops are wide and flat, perpendicular to the disks, so as to lift the meal from the bottom of the dryer to the outlet.

Four types of scoops are used in the dryer: neutral scoops for brewing, positive scoops for meal advancement, negative scoops to improve drying, and extraction scoops to push and extract meal from the meal dryer.

The vapors evaporated from the meal exit at the top of the dryer (60). They are condensed in a plate heat exchanger with cold water. This water is cooled in an air cooler located on the roof of the transformation building. At the condenser outlet, the condensates are separated from the non-condensed vapors and either reused or sent to the wastewater system.

The non-condensed vapors are drawn off by a vacuum pump, which creates a vacuum in the dryer. The vacuum is 400 mbar. At this pressure, the boiling temperature of the water is 75° C. This ensures a relatively low temperature in the dryer to preserve the meal's qualities, and, in particular, its digestibility.

The dryer (60) is isolated from upstream and downstream equipment that is not under vacuum. Upstream of the dryer (60), isolation is provided by the two valves, which cannot be opened simultaneously. Downstream, a sluice provides a watertight seal.

### Cooling the Meal

The dryer outlet is by gravity and not by a screw as in the prior art, to limit the risk of condensation and bacterial contamination, as well as the risk of clogging.

Meal exiting the dryer (60) falls into a sluice and then into a heat-insulated pneumatic conveyor (63), which conveys the meal to the cooler (64). Transfer to the dryer outlet is by pneumatic conveying (63) and not by a screw as in the prior art, to limit the risk of condensation and bacterial contamination. The meal is cooled to room temperature plus 10° C.

Ambient air is drawn in around the cooler (64) and circulates in counter-current through the meal layer. The warm air is filtered and discharged outside the building.

Once a layer of meal has cooled to the target temperature, flaps located beneath the meal layer open automatically to allow the meal to flow by gravity to the mill (65) located beneath the cooler.

### Structure of the Mill (65)

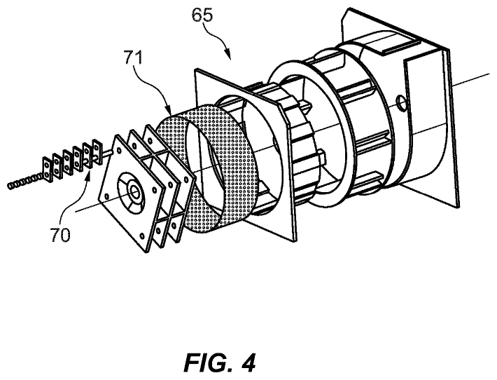

The meal is ground in a hammer mill (65) shown in FIG. 4. Hammers (70) located on a rotor knock the meal through a grid (71) with a mesh size of 2 mm, for example. The grain size of the meal at the outlet of the mill is thus, for instance, less than 2 mm.

Magnets are fitted to the feed of the mill to protect the mill from damage caused by metallic foreign bodies. At the end of each production run, the operator can dismantle and clean the magnets, then replace them. Removing and cleaning the magnets makes it possible to:
assess whether metallic materials have been stopped, and if so, quickly identify the causes upstream of the process; and
prevent the magnet from becoming “full” and losing its effectiveness.

The mill fan (72) conveys the meal pneumatically to the 3 m³ buffer hopper (66) located downstream. Upstream of this hopper, on the pipe, a second magnet is installed to capture any metal particles generated during grinding.

Packaging the meal

The meal is packaged in 500 kg “big-bag” (trade name) containers (67), suitable for food contact. A filling station is located beneath the 3 m³ buffer hopper (66). This buffer hopper (66) stores meal while the operator changes containers (67). The filling station is on load cells, which are wired to the programmable logic controller.

The operator first installs an empty container (67) on the filling station, then starts inflating the container (67). Once inflation is complete, the operator presses another button to extend the container (67), then a final button that signals to the central programmable logic controller that the container (67) is ready to receive the meal.

The filling sequence for the container (67) is then started automatically. The target weight setpoint entered into the programmable logic controller is 500 kg. The empty container (67) is tared, then the sluice at the bottom of the buffer hopper and the hopper's vibrating bottom start up. Filling stops when the target weight is reached (the sluice and vibrating bottom stop). The operator retrieves the container (67), and transports it to the finished product storage area. That operator can then install a new container (67) if any meal remains in the buffer hopper (66).

It is also suggested to store the meal in bulk in silos and market it in bulk trucks.

### Managing the Glue Water

The glue water produced by the three-phase decanter (12) is made up of the water contained in the larvae, any water added in the method (residue from dewatering during slaughtering and/or dilution of the larvae with method hot water), plus a little fat and dissolved solids.

The recovery of glue water as a dilution medium is covered here. The main advantages are reduced dilution water consumption and energy savings, as it does not need to be heated to 80° C.

As separation by decantation is not perfect, some of the solids and fats end up in the glue water. The solids that pass through the glue water are often good-quality, highly soluble proteins. There is therefore a strong interest in recycling them to increase overall line output and improve meal quality.

As described above, part of the glue water is recycled upstream of the separation method to optimize separation and minimize hot water consumption.

Alternatively, a small amount of glue water can be fed directly into the dryer to be dried in combination with the cake. This step is optional and not necessary for the method to run smoothly.

In general, the glue water is fed to a two-effect evaporator (50) (i.e., with two evaporation stages in two specific plate heat exchangers). The evaporator (50) enables the dry matter percentage to be increased from 5% to between 30% and 50%. In the first effect, the glue water is pre-concentrated. Then, in the second effect, the concentration reaches the target value. Concentration is estimated by measuring density, which is used as a sign of dry matter quantity.

### Energy Optimization of Evaporation

To ensure the concentration of the glue water, a significant quantity of water is evaporated. Energy must therefore be brought in.

The evaporator (50) has been designed to operate with two possible energy sources:
live steam; and
recovery of vapors (i.e., water evaporated from glue water), mechanically recompressed to provide more energy.

Mechanical vapor recompression is possible as soon as evaporative vapor production exceeds the minimum load point (“turndown”) of the compressor, known as MVR (Mechanical Vapor Recovery). Before this point, live steam is used to heat the 1^{st} effect, and the vapors extracted from the 1^{st} effect heat the 2^{nd} effect. Beyond this point, the vapors from the 1^{st} and 2^{nd} effects are recovered and recompressed to provide the required energy. In MVR mode, no live steam is used except at evaporator (50) start-up.

Switching between MVR and live steam mode is done by turning a set of spectacle blinds to modify the steam and vapor circuits. The switch therefore necessarily takes place when production is not underway. These two modes enable the evaporator (50) to be operated over a wide range of glue water flow rates.

Optimizing product quality

In order to limit the time/temperature couple applied to the glue water, the evaporator (50) is evacuated so that evaporation takes place at a temperature below 100° C. A vacuum pump maintains a vacuum of 400 mbar in the exchangers to ensure a temperature of around 75° C. during the method.

### Recovering the Concentrate

After evaporation, the glue water is concentrated to around 30-50% dry matter to form a concentrate. The concentrate is pumped by a pump (51) from the evaporator concentrate storage tank to the dryer feed lock. The concentrate is transferred only when the three-phase decanter is in operation. As the concentrate is a sticky product, it cannot be dried on its own in the disc dryer, as this may clog the discs. A maximum of 20% concentrate is added to the cake to prevent clogging.

### Oil Production

The oil leaving the three-phase decanter flows from the bowl (30) through the pipe (34) by gravity to a small 100 L buffer tank (40), from where it is drawn off and transferred by a pump (41) to a storage tank (44) with a capacity of 30m³. It is then packaged in flexible or rigid intermediate bulk containers (IBCs) (47), or shipped by tanker truck fed by an eccentric rotor pump.

### Oil Quality

At the pump discharge (41), a turbidity sensor ensures that the oil sent to the storage tank (44) is pure. This probe measures turbidity, i.e., opacity, to obtain a moisture value. When the turbidimeter detects a moisture content above a threshold of between 0.3% and 1%, the oil is recycled to the tank (11) upstream of the three-phase decanter (12).

The water-laden oil is recycled upstream of the three-phase decanter, where it is mixed with the ground larvae pulp in the agitated tank (11), before being separated again in the decanter.

A three-way valve (43) controlled by the turbidimeter (42) transfers the oil either to the storage tank (44) or to the oil recycling tank (11).

When the line starts up, the three-way valve (43) is open in the oil recycling position to the tank (11) and closed to the storage tank (44): Before the decanter is stabilized in the three-phase decanter (12), the oil is loaded with water and therefore recycled via the buffer tank (11) and not sent to the storage tank (44).

When the oil is pure, the three-way valve (43) closes toward the tank (11) and opens toward the oil storage tank (44).

A magnet (46) and a filter trap any metallic foreign bodies at the feed point of the storage tank (44), or downstream of the storage tank at the IBC packaging point or tanker.

At the end of production, compressed air is injected at the pump discharge (41). This pushes the oil toward the storage tank (44) to empty the pipes.

### Oil Temperature Maintenance

The buffer tank (40) at the outlet of the three-phase decanter (12) is thermally insulated. All oil pipes are electrically traced and insulated to ensure that the oil remains liquid in the pipes and does not congeal. The oil congeals at a relatively low temperature, around 30° C.

The storage tank (44) is maintained at temperature by a double jacket wherein hot water circulates without contact with the product. Temperature maintenance prevents the oil from congealing in the tank (44).

A hot water loop circulates in the tank's double jacket. The water, at a temperature of 58° C., is heated in a plate heat exchanger using water at 60° C., and circulates continuously in the circuit via a pump.

### Oil Packaging

A conditioning pump (45) draws oil from the 30 m³ storage tank (44) and transfers it to a 1 m³ IBC (47). The IBC containers used to package the oil are suitable for food contact.

An operator sets up an empty IBC container on a pallet truck with built-in scales, tares it, and then places a hose connected to the packaging pump's discharge (45) into the container.

The conditioning pump (45) operates at a flow rate of 2 m³/h, so filling an IBC (47) takes 30 minutes. The first operator checks the weight of the IBC (47) on the scale. Alternatively, a second pump can be used, drawing from the storage tank and delivering to a tanker truck. A flexible hose is connected to the discharge of this pump to the tanker truck parked outside the building.

### Adding Antioxidant to Oil

To avoid the appearance of a rancid odor or taste, and to guarantee the oil's good shelf life, antioxidant is added to the oil's IBCs (47), notably an antioxidant of the tocopherol type.

The antioxidant concentration in the oil is set at 800 ppm. When starting to fill the container with oil, the operator validates the total quantity of antioxidant to be added for a container filled with oil. The antioxidant pump then automatically pumps the antioxidant from the canister into the container at the same time as the container is filled with oil, to promote the mixing and homogeneity of the finished product.

Alternatively, the oil is sent into the IBC container. Once finished, the oil is weighed into the container and the antioxidant is added directly to the container depending on the quantity of oil weighed and the desired concentration. The mixture is then stirred to mix the antioxidant.

In the same way, antioxidant is added to the oil piping when filling a tank truck.

1. An installation for treating arthropod larvae, comprising at least one piece of equipment producing glue water, which is extracted from a mixture of ground larvae, and a glue water circuit supplying a ground larvae treatment equipment comprising a circuit for diluting the ground larvae and/or a glue water evaporator and/or a meal dryer.

2. The installation of claim 1, further comprising a decanting system for separating the solid and liquid phases of the ground larvae, having three outlets including:
the a solid phase outlet is connected to a meal dryer;
a first liquid outlet connected to the glue water circuit; and
a second liquid outlet connected to an oil collection circuit.

3. The installation for of claim 2, wherein the solid phase outlet is directly connected to a meal dryer via a chute located below the outlet of the decanting system, allowing transfer of the solid phase from the decanting system to the meal dryer through the chute by gravity.

4. The installation of claim 2, wherein the meal dryer comprises a vacuum chamber.

5. The installation of claim 4, further comprising, upstream from the meal dryer, a sealed transfer lock formed by two valves that open and close alternately to maintain a vacuum in the vacuum chamber of the meal dryer.

6. The installation of claim 4, further comprising a sealed isolation sluice for the meal dryer, the meal drying being maintained under vacuum.

7. The installation of claim 2, herein the meal dryer has disks at an outlet of the meal dryer with extraction scoops for directing meal toward the outlet of the meal dryer.

8. The installation of claim 2, wherein the meal dryer is supplied with concentrate from a glue water evaporator and/or glue water from the three-phase separation decanting system (12), as well as with an antioxidant injection line.

9. The installation of claim 2, further comprising a condenser comprising a plate exchanger for condensing vapors evaporated from the meal during drying, and a mechanism configured to discharge condensates from the condenser.

10. The installation of claim 2, further comprising equipment for concentrating glue water to 30% to 50% dry matter to form a concentrate reinjected upstream of a meal dryer, and a mixer configured to mix the concentrate with cake from the decanting system.

11. The installation of claim 2, further comprising a three-way valve having an inlet connected to the second liquid outlet, one outlet of the three-way valve being connected to a tank upstream of the decanting system, and another outlet of the three-way valve being connected to a buffer tank, the installation further comprising a means for measuring a quality of oil coming from the decanting system, and for delivering a signal for controlling the valve.

12. The installation of claim 11, wherein the means for measuring the quality of the oil is a turbidimeter.

13. The installation of claim 2, further comprising thermal regulation means for ensuring that a temperature of the ground larvae at an inlet to the decanting system is between 80° C. and 95° C.
